(19) United States
(12) Patent Application Publication
(10) Pub. No.: US 2026/0112505 A1
(43) Pub. Date: Apr. 23, 2026

(54) Machine-Learned Model Architecture for Generating Individual-Specific Instructions

(71) Applicant: OPTUM, INC., Minnetonka, MN (US)

(72) Inventors: Bilikis J. Oladimeji, Newton Upper Falls, MA (US); Reem A. Hussain, Silver Spring, MD (US); Aaron C. Wacker, Mound, MN (US)

(21) Appl. No.: 18/924,533

(22) Filed: Oct. 23, 2024

(51) Int. Cl. G16H 50/70; G16H 70/20

(52) U.S. Cl. CPC G16H 50/70; G16H 70/20

(57) ABSTRACT

Systems and methods for using generative language models to generate individual-specific instructions are disclosed herein. When receiving a request for instructions for a condition, the disclosed techniques map the condition to a condition concept of an ontological representation, map individual-specific information to an individual-specific concept of the ontological representation, determine from the ontological representation whether the individual-specific concept is a comorbidity concept with respect to the condition concept, and generate a prompt based on the request for instructions and on property data associated with the condition concept and the comorbidity concept. The disclosed techniques generate the requested instructions by applying the prompt to a generative language model.

### TECHNICAL FIELD

The present disclosure generally relates to automatic generation of instructions, and more particularly, to using generative language models to generate individual-specific instructions for patients.

### BACKGROUND

Following utilization of healthcare services, patients who are managed for acute or chronic conditions, including medical procedures, desire simplified and easily accessible information about their condition to be active participants in their recovery or management. Patient education after a medical visit is often fragmented and provided by different members of the care team. Moreover, health care teams are often short-staffed and lack the time to communicate individual-specific care instructions for patients. The care teams frequently rely on patient educational materials from paid curated resources or open-source resources. Traditionally, these patient education materials have been static and generic, not taking into account the individual characteristics, health conditions, or specific needs of the patient. This one-size-fits-all approach often results in materials that may not fully engage patients or adequately address their unique questions and concerns. Regardless of how a patient's health state may differ from the average/typical, the patient materials always display the same information for each patient with a similar condition. Further still, the patient materials are decontextualized, with different patients receiving the same information regardless of where the patients are on their health journeys.

Accordingly, a need exists for generating individual-specific instructions in an accurate, contextualized, and efficient manner.

### BRIEF DESCRIPTION OF THE DRAWINGS

The figures described below depict preferred embodiments for purposes of illustration only. One skilled in the art will readily recognize from the following discussion that alternative embodiments of the systems and methods illustrated herein may be employed without departing from the principles of the disclosure described herein.

FIG. 1 depicts an example computing system in which various embodiments of the present techniques are implemented, according to one aspect.

FIG. 2A depicts an exemplary process for training and tuning a clinical language model, according to some aspects.

FIG. 2B depicts an exemplary process for processing a clinical knowledge base into an ontological representation and embeddings, according to some aspects.

FIG. 2C depicts an exemplary process for linking patient information to an ontological representation and embeddings and extracting relevant information, according to some aspects.

FIG. 2D depicts an exemplary process for providing a prompt to the trained clinical language model and receiving responsive instruction output, according to some aspects.

FIG. 3 depicts an exemplary medical ontological representation, according to some aspects.

FIG. 4 depicts an exemplary medical knowledge vector database, according to some aspects.

FIGS. 5A and 5B depict an exemplary computer-implemented method for generating responsive instructions, according to some aspects.

### DETAILED DESCRIPTION

### Introduction

The techniques disclosed herein generate individual-specific instructions in an accurate, contextualized, and efficient manner. For example, the methods and systems can create personalized messages to patients providing a summary of their health, information on their clinical condition, and/or guidance for medication use or other interventions (e.g., post-operative instructions, insulin titration, correct use of inhaler device, wound care, etc.). In some embodiments, the methods and systems generate dynamically updated instructions pursuant to changes in the patient's condition. The disclosed techniques provide a generative language model (e.g., a large language model (LLM) or generative pre-trained transformer (GPT)) with individual-specific information and a prompt requesting that the generative language model produce instructions or answer a question using the provided information. With the provided information, the generative language model can answer questions and provide instructions that are specific to the individual and the current state or situation of the individual. The prompt includes individual-specific information (e.g., via retrieval augmented generation (RAG)) that the system or method deems relevant.

The disclosed techniques have certain advantages over alternative techniques, such as the alternative technique of using a general-purpose language model that relies solely upon knowledge obtained from conventional training methods. For example, because the language models of such alternative techniques are not trained upon a particular field of knowledge (e.g., a medical field), such models are generally unable to provide detailed instructions or answer questions pertaining to that particular field, or the language models may hallucinate and give inaccurate output. Moreover, such language models lack knowledge of individual-specific information, as well as information arising after the language model is trained. Merely tuning a generative language model with domain-specific knowledge does not address all these disadvantages in a satisfactory manner. For example, tuning the generative language model can be computationally expensive and requires high-quality training data. Moreover, it may be impractical to repeat such a tuning process for each individual's specific information or for newly obtained domain-specific knowledge.

Another advantage of the disclosed techniques stems from mapping conditions (e.g., as indicated by requests for patient instructions) and individual-specific information (e.g., as reflected in medical records) to concepts in an ontological representation, such as a knowledge graph. The ontological representation can link concepts from disparate knowledge data sources, e.g., clinical practice guidelines, educational materials, etc. Using the ontological representation, the disclosed techniques can efficiently and accurately determine which individual-specific concepts are relevant to which condition concepts. For example, relevancy may be determined by analyzing whether a “path” comprising intervening concepts (e.g., nodes of the representation) and relationships (e.g., edges of the representation) exists between the individual-specific concepts and the condition concepts.

In contrast, without the disclosed use of an ontological representation, determining a relationship between disparate concepts may rely solely upon techniques such as determining semantic similarity by computing distances between embeddings (high-dimensional vectors). Such techniques can require a far greater volume of knowledge base information, encoded into embeddings, to establish the semantic similarity between the condition and the individual-specific information. Thus, the disclosed techniques can provide greater computational/processing efficiency over alternative approaches.

Further, it can be difficult to troubleshoot faulty instruction output generated by pure distance/semantic similarity techniques, at least because visualizing embeddings and associating those embeddings to text is challenging. Conversely, if the trained language model of the disclosed techniques provides incorrect or incomplete instructions (e.g., during testing), an administrator can query the trained language model to learn about the selected path(s) and thereby enable debugging and remediation steps.

Thus, the disclosed techniques include improvements in computer functionality at least because the techniques improve the accuracy, relevance/context, and efficiency of language model queries and response generation.

In some embodiments, the disclosed techniques encompass additional steps operations that further enhance utility and effectiveness. These can include, for example, retrieving and normalizing concepts from educational resources, generating the ontological representation, and incorporating citations from the educational resources into the generated instructions. In some embodiments, the techniques receive user questions and generate answers to these questions using the generative language model. This interactive aspect of the techniques ensures that individuals are not just passive recipients of information but are actively engaged in understanding and applying the instructions to their condition.

In some embodiments, the disclosed techniques automate data abstraction to render individual records machine readable and computable. Condition information and individual-specific information are abstracted into normalized concepts and linked to knowledge base concepts. In some embodiments, using a combination of the abstract representation of the condition and individual-specific information and normalized and harmonized knowledge, the systems and methods generate an automatic prompt for use with a generative large language model, which eliminates the need for the user to explicitly “ask” the generative language model to generate instructions. As a result of automatic prompting, the user is not required to list our relevant points from the individual's biography or record—this information can instead be automatically updated. The automatic prompting ensures that the correct individual record is referenced and linked to the pertinent knowledge base concepts pertaining to the individual's unique status.

Of course, it should be appreciated that the advantages and technical improvements described above and elsewhere herein are not the only advantages and/or technical improvements that may be realized because of the techniques described herein. Other advantages and/or technical improvements to the functioning of a computer itself or other technologies or technical fields may be apparent to one of ordinary skill in the art. Moreover, while described herein primarily in the medical/health care context, the disclosed techniques may be readily applied in any other suitable field for any suitable purpose.

### Example Computing System

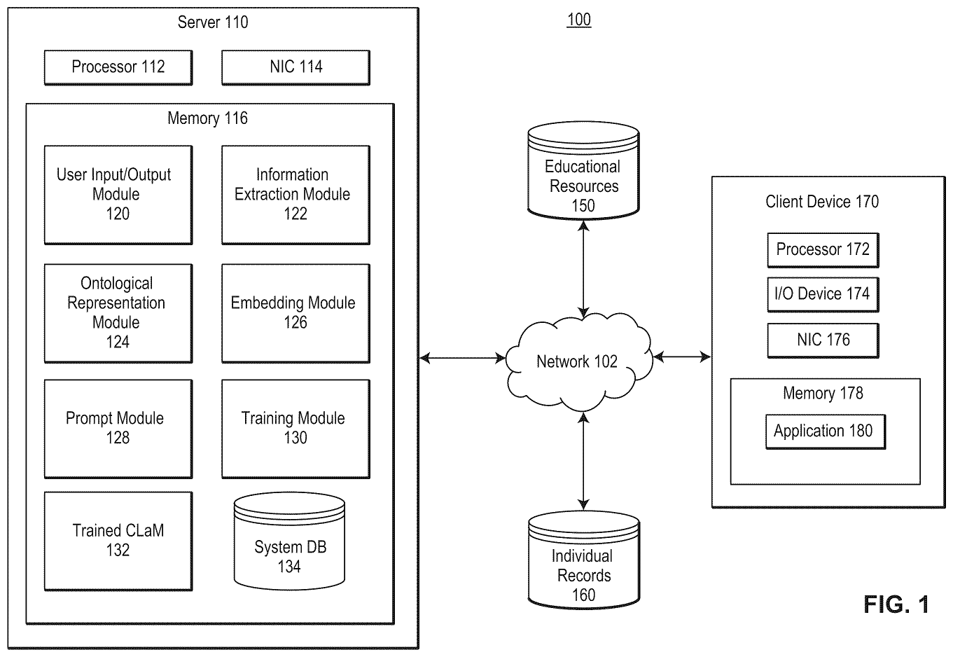

FIG. 1 depicts an example computing system 100 in which various embodiments of the present disclosure may be implemented. Although FIG. 1 depicts certain entities, components, equipment, and devices, it should be appreciated that additional or alternate entities, components, equipment, and devices are also possible. In some embodiments, the example computing system 100 generates personalized patient instructions (e.g., as answers to patient questions) based on individual health records and condition-specific information. Of course, it should be appreciated that, while the various components of the example computing system 100 (e.g., network 102, server 110, system database 140, educational resources repository 150, individual records repository 160, client device 170, etc.) are illustrated in FIG. 1 as single components, the example computing system 100 may include a plurality of servers 110, system databases 140, educational resources repositories 150, individual records repositories 160, and/or client devices 170 that are simultaneously connected to the network 102 at any given time. In some embodiments, one or more components of the computing system 100 (e.g., server 110, educational resources repository 150, individual records repository 160, etc.) are located in a remote data center, such as a cloud computing environment.

The network 102 connecting these components includes one or more communication networks of one or more types, such as local area networks (LANs), wide area networks (WANs) like the Internet, wired, and/or wireless, or a combination thereof. In one embodiment, for example, the network 102 includes a wireless cellular network (e.g., 4G, 5G, 6G, etc.). Generally, the network 102 enables bidirectional communication between the server 110 and the system database 140, educational resources repository 150, the individual records repository 160, and the client device 170.

In one embodiment, the network 102 comprises one or more cellular base stations, such as cell tower(s), communicating with one or more other components of the computing system 100 via wired/wireless communications based upon any one or more of various wireless communication standards, including NMT, GSM, CDMA, UMTS, LTE, 5G, 6G, or the like. Additionally or alternatively, the network 102 may comprise one or more routers, wireless switches, and/or other nodes communicating with the components of the computing system 100 based upon any one or more of various other wireless and/or wired communications standards, including by non-limiting example, IEEE 802.11a/ac/ax/b/c/g/n (Wi-Fi®), Bluetooth®, Ethernet, and/or the like.

The server 110 performs at least some of the functionalities and techniques disclosed herein. In one embodiment, the server 110 is a server of a healthcare provider. The server 110 includes a processor 112, a network interface 114, and a memory 116. As used herein, the term “server” encompasses single servers as well as multiple servers that are co-located or remotely distributed. The server 110 may be part of a cloud network or may otherwise communicate with other hardware or software components within one or more cloud computing environments to send, retrieve, or otherwise analyze data or information described herein. In some example embodiments, the computing system 100 comprises, or exists within, an on-premises computing environment, a multi-cloud computing environment, a public cloud computing environment, a private cloud computing environment, and/or a hybrid cloud computing environment.

The server 110 includes a processor 112, which may include any number of processors and/or processor types, such as central processing units (CPUs) and graphics processing units (GPUs). In some embodiments, the processor 112 also includes one or more field programmable gate arrays (FPGAs), and/or any other suitable processor(s). The processor 112 is communicatively coupled to a memory 116 (e.g., via a computer bus not depicted in FIG. 1), such that the processor 112 can execute machine-readable instructions stored in memory 116 to perform operations of the server 110 disclosed herein.

The server 110 includes a network interface 114, which may include any suitable network interface controller(s) and facilitates communication over the network 102 between the server 110 and other components. The network interface 114 communicates via any suitable wired and/or wireless connection (e.g., according to any of the wired or wireless protocols/standards discussed above for network 102 and using any suitable controller(s) of the network interface 114).

The memory 116 may include volatile and/or non-volatile memory, such as read-only memory (ROM), electronic programmable read-only memory (EPROM), erasable electronic programmable read-only memory (EEPROM), random access memory (RAM), hard drives, solid state drives, flash memory, MicroSD cards, and/or other type(s) of memory. The memory 116 may also store an operating system (e.g., Microsoft Windows, Linux, UNIX, etc.) capable of facilitating the functionalities, applications, methods, or other software as discussed herein. In some embodiments, the memory 116 stores a plurality of software modules including respective sets of computer-executable instructions. These software modules include one or more of a user input/output module 120, an information extraction module 122, an ontological representation module 124, an embedding module 126, a prompt module 128, a training module 130, and a trained clinical language model (CLaM) 132. The software modules may be implemented in any desired programming language, and may be implemented as machine code, assembly code, byte code, interpretable source code, or the like (e.g., via Python, C, C++, Java, JavaScript, etc.). The software modules support operations related to generating personalized patient instructions.

The server 110 includes or has access to a system database 134. One or more of the above-noted software modules may store and/or retrieve information from the system database 134. The system database 134 may include a relational database, (such as Oracle®, IBM DB2®, or MySQL), a NoSQL based database (such as MongoDB), a vector database (such as Microsoft Azure Cosmos DB® or Pinecone), and/or a graph database (such as Neo4j), for example.

The server 110 includes or has access to the educational resources repository 150. In some embodiments, the educational resources repository 150 includes one or more web servers, file servers, database servers, or any other suitable information storage servers. The educational resources repository 150 generally stores medical education information, such as medical treatises, dictionaries, journal papers, guidelines, procedures, lectures, clinical practice guidelines, standard operating procedures, known drug interactions, etc. comprising text, images, audio, and/or video. The educational resources repository 150 may be owned or operated by the operator of the server 110 or one or more third-party organizations.

The server 110 also includes or has access to the individual records repository 160. The individual records repository 160 includes medical records and/or medical insurance claims, including health summaries, healthcare utilization data, claims records, prescriptions, diagnoses, laboratory test results, risk adjustment factor (RAF) score or other risk scores, vital sign information, past hospitalizations and surgeries, etc. The individual records repository 160 stores the medical records and/or insurance claims in a structured or unstructured format. In some embodiments, the individual records repository 160 includes a relational database, a NoSQL based database, and/or other suitable database(s). The individual records repository 160 may be owned or operated by the operator of the server 110 or one or more third-party organizations.

In some embodiments, the client device 170 includes one or more computing devices (e.g., desktop computer, laptop computer, terminal), mobile devices, wearables, smart watches, smart contact lenses, smart glasses, AR glasses/headsets, VR glasses/headsets, mixed or extended reality glasses/headsets, and/or other suitable electronic or electrical components. The client device 170 includes a memory 178 and a processor 172 for, respectively, storing and executing one or more software modules, such as an application 180, computer-executable instructions, operating systems, etc. In some embodiments, the memory 178 includes one or more suitable storage media such as a magnetic storage device, a solid-state drive, RAM, and/or any other type of volatile or non-volatile memory. The client device 170 includes an input/output device 174, which includes a display (dedicated or touchscreen). The input/output device 174 is configured to present graphical user interfaces (GUIs) and/or other information for presentation to a user and/or receive input from the user. The client device 170 includes a NIC 176, which accesses services or other functionalities and/or components of the computing system 100 (e.g., of server 110) via the network 102. In some embodiments, the computing system 100 includes multiple client devices similar to the client device 170. In other embodiments, however, the computing system 100 excludes client device 170. For example, the server 110 may instead operate without providing any information to, and/or without receiving any information from, an external device or system.

The client device 170 includes an application 180, which is generally used to request or receive information/data from and/or provide information/data to, the server 110. In some embodiments, the application 180 includes a web browser, a client application, and/or a mobile application. The application 180 includes a GUI for presenting controls and information on the input/output device 174. In one embodiment, the client device 170 is associated with a medical professional (e.g., healthcare provider). In another embodiment, the client device 170 is associated with a patient seeking answers to questions via a website or an interactive chat session.

Returning to the server 110, the memory 116 stores a user input/output module 120, which receives input from and provides output to users. In some embodiments, the user input/output module 120 is configured to receive a patient condition and a request for instructions from medical personnel and output personalized patient instructions which the medical personnel can then forward to or otherwise share with the patient. In some embodiments, the user input/output module 120 directly receives the requests (e.g., free-form text questions) from, and provides the answers to, a patient user via a website or an interactive chat session. In some embodiments, the user input/output module 120 implements a condition(s)-to-goal(s)-to-intervention model. In this model, if a patient has N conditions and wants to affect at least one of those conditions with goals, the user input/output module 120 places those conditions in-scope, and provides educational materials on optional interventions that the patient can review and possibly implement to treat the in-scope conditions. The user input/output module 120 may implement user authentication, such as username/password, one-time password, biometrics, etc. The client device 170 accesses the server 110 via the application 180 communicating remotely with the user input/output module 120 over network 102.

In the example computing system 100, the information extraction module 122 extracts information from medical resources. In particular, the information extraction module 122 requests and receives, and/or scrapes, medical knowledge resources from the educational resources repository 150 and patient medical information from the individual records repository 160. In some embodiments, the information extraction module 122 performs optical character recognition (OCR) on retrieved scanned documents to generate text data. In some embodiments, the information extraction module 122 transcribes audio and/or video resources into text data. The information extraction module 122 may split text into a plurality of chunks, for use by the embedding module 126 as discussed below. The information extraction module 122 may perform named entity recognition (NER) to identify and extract the names of diseases, conditions, procedures, symptoms, prescriptions, and/or other entities.

The ontological representation module 124 generates, updates, and queries a medical ontological representation. The ontological representation module 124 generates the medical ontological representation from a standardized medical coding scheme, such as Fast Healthcare Interoperability Resources (FHIR), Systematized Nomenclature of Medicine-Clinical Terms (SNOMED-CT), Logical Observation Identifiers Names and Codes (LOINC), International Classification of Diseases (ICD), etc. The ontological representation module 124 may supplement the medical ontological representation with medical knowledge retrieved from the educational resources repository 150. The ontological representation module 124 saves the medical ontological representation into the system database 134, in some embodiments. The medical ontological representation represents/includes a plurality of medical concept types, such as a disease, condition, prescription, procedure, etc. A medical concept is associated with one or more properties, such as a type of the medical concept, a standardized identifier for the medical concept, a source citation for information associated with the medical concept, a corresponding embedding for the medical concept, one or more synonyms of a term or phrase of the medical concept, one or more clinical characteristics of the medical concept, and/or other suitable properties. The medical ontological representation also represents/includes a plurality of relationships. A “relationship,” as the term is used herein, is a direct link or connection between two nodes. A relationship is associated with one or more properties, such as the relationship type, standardized identifier, etc. In some embodiments, the ontological representation module 124 queries the medical ontological representation using a graph query language, such as Cypher®. The ontological representation module 124 determines a concept in the medical ontological representation corresponding to a patient condition and a concept corresponding to patient information. In some embodiments, the ontological representation module 124 generates a visual representation of a portion of the medical ontological representation using a diagramming language, such as Mermaid.

The embedding module 126 abstracts text chunks from the information extraction module 122 into a plurality of embeddings. The embedding module 126 converts the medical knowledge text chunks into embeddings. In some embodiments, the embedding module 126 implements a text embedding technique, such as Word2Vec or Doc2Vec. The embedding module 126 saves the embeddings into a vector database in the system database 134 in some embodiments. In some embodiments, the embedding module 126 computes a vector search query, such as cosine similarity or Euclidean distance, between two specified embeddings to estimate the semantic similarity of the concepts represented by the embeddings.

The prompt module 128 generates a prompt and submits the prompt to a generative language model, such as the trained CLaM 132. The prompt module 128 includes directives to the generative language model in the prompt. In some embodiments, the prompt module 128 uses generation augmented retrieval (GAR) to augment the prompt with contextual facts and relationships. Alternatively or additionally, the prompt module 128 can use retrieval augmented generation (RAG) to include clinical information, individual-specific comorbidity information, and/or other relevant information in the prompt. The prompt module 128 receives the output of the generative language model generated in response to the prompt.

The training module 130 generates a trained CLaM 132, the defining parameters of which (weights, hyperparameters, etc.) may be stored in memory 116. In some embodiments, the training module 130 trains the language model using standardized medical guidelines and patient instructions. The trained CLaM 132 outputs patient instructions and answers in prose text, linking unique patient risk factors and past clinical history to the medical condition. In some embodiments, the trained CLaM 132 receives chat prompts and outputs chat responses. In some embodiments, the trained CLaM 132 adjusts the patient instructions and the chat responses to the educational level of the patient. In some embodiments, the trained CLaM 132 include citations to medical educational resources in the generated instructions and answers. Training and operation of the trained CLaM 132 is discussed in more detail below.

### Example Processes

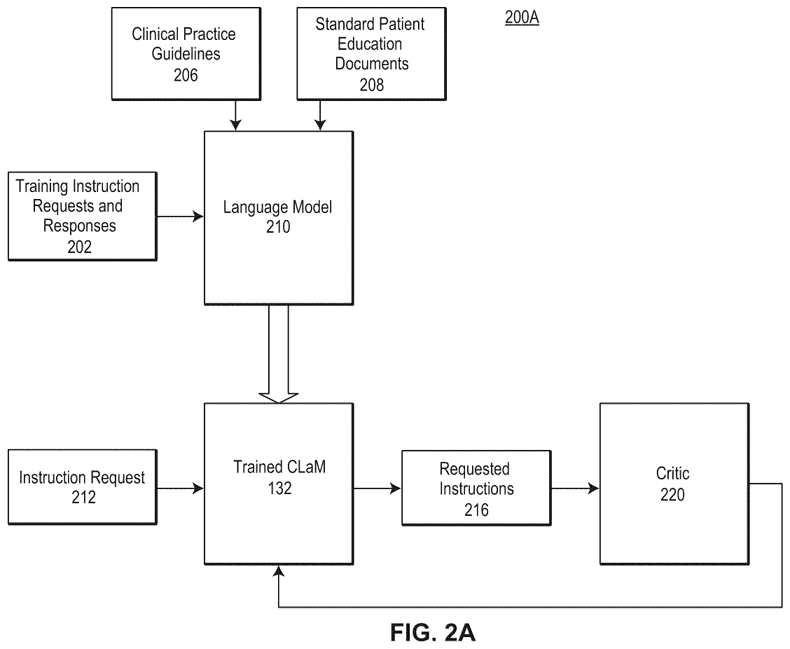

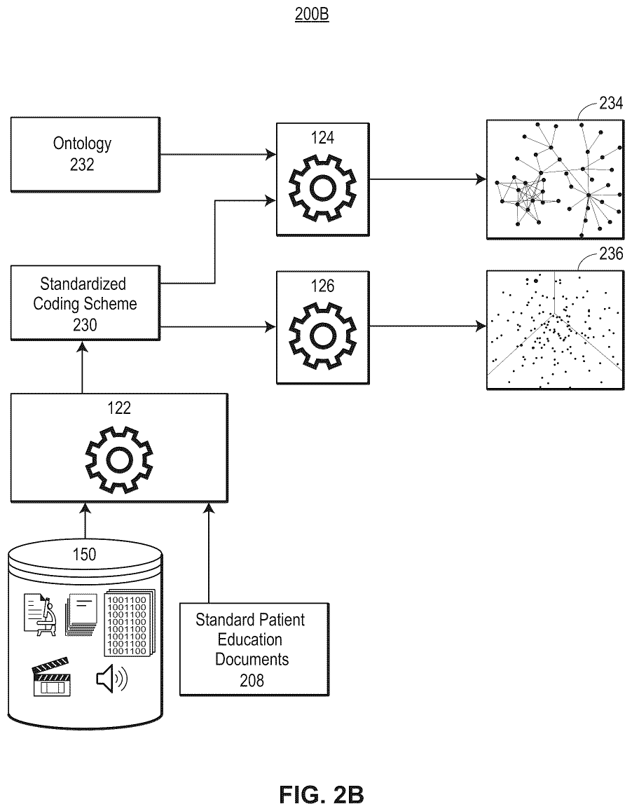

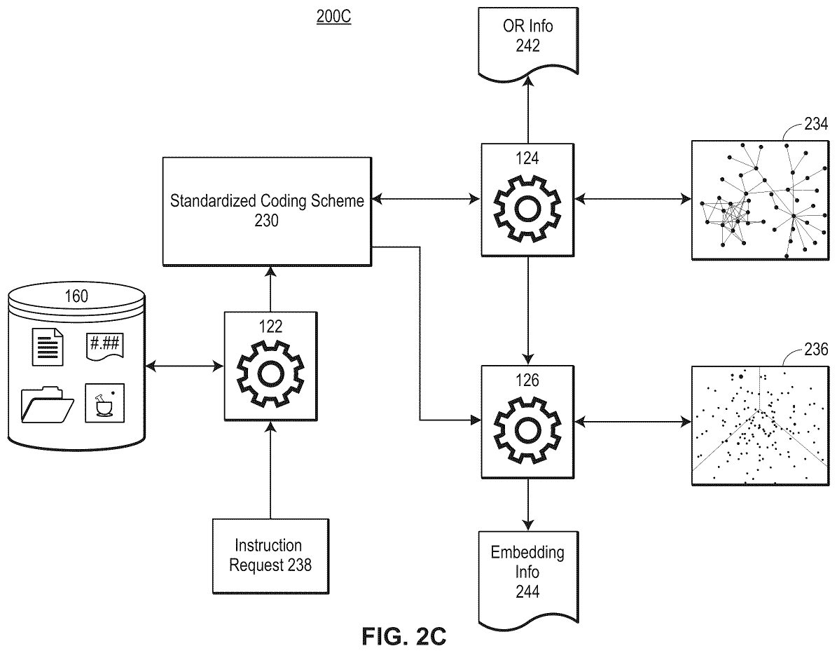

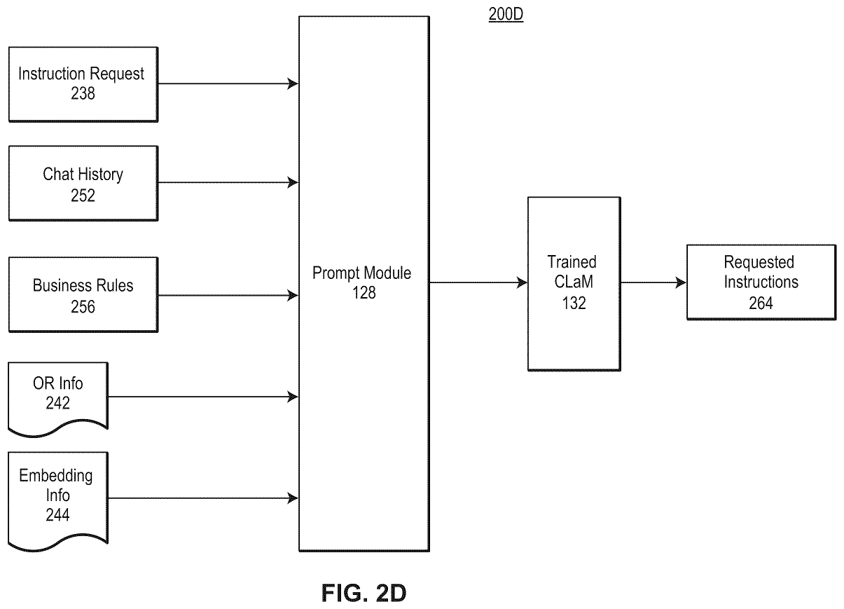

FIG. 2A depicts an example process 200A for training a language model 210 and tuning a trained CLaM 132. In alternative embodiments, the process 200A includes more, fewer, and/or different components and/or operations than those shown in FIG. 2A. In some embodiments, the training and tuning is performed by the training module 130. The training module 130 generates a trained CLaM 132 from a language model 210. In some embodiments, the language model 210 includes a general-purpose, transformer-based large language model (LLM), such as GPT-4 or Llama.

In some embodiments, the training module 130 trains the language model 210 with training instruction requests and responses 202. The training instruction requests and responses 202 include pairs of instruction requests and corresponding model responses. For example, one training instruction prompt may be “Write instructions for post-operative care of a knee surgery patient” or “When should I resume taking Warfarin after my knee surgery?” and the corresponding model responses may include text instructing the requesting person as to how to care for the identified condition in the identified context.

In some embodiments, the training module 130 trains the language model 210 with clinical practice guidelines 206. The clinical practice guidelines 206 may include medical guidelines and/or rules for patient care, such as instructing the patient to call 911 if they are experiencing symptoms of a heart attack. In some embodiments, the training module 130 trains the language model 210 with standard patient education documents 208, such as non-personalized patient instructions.

After training, the language model 210 becomes the trained CLaM 132. In some embodiments, further tuning and validation steps are performed on the trained CLaM 132. In operation, the trained CLaM 132 is prompted with an instruction request 212, and the trained CLaM 132 generates one or more sets of instructions 216 as output. At the reinforcement learning stage, a critic 220, such as a human reviewer, rules-based agent, adversarial model, and/or an additional language model, may act as an answer evaluator by reviewing the one or more sets of requested instructions 216 and providing feedback. In some embodiments, the critic 220 assigns a rank or numerical score to the instructions 216. The training module 130 uses the ranks or scores to fine tune the trained CLaM 132 by rewarding more correct and/or complete instructions and penalizing incorrect and/or incomplete instructions.

FIG. 2B depicts an example process 200B for generating a medical ontological representation 234 and a medical knowledge vector database 236. In alternative embodiments, the process 200B includes more, fewer, and/or different components and/or steps than those shown in FIG. 2B. The standardized coding scheme 230 may include one or more standardized medical term coding schemes, such as FHIR, SNOMED CT, LOINC, ICD, etc. In some embodiments, the standardized coding scheme 230 includes a thesaurus comprising alternative, equivalent names for medical conditions, diseases, drugs, etc. In some embodiments, the standardized coding scheme 230 is organized as a taxonomy with classes and subclasses. In some embodiments, the standardized coding scheme 230 includes a tree structure linking medical conditions to treatments. For example, one tree may include “Infectious Diseases->Cholera->Antibiotic Treatment.” Another exemplary tree may include “Neoplasms->Malignant Neoplasms of Lip, Oral Cavity, and Pharynx->Chemotherapy->Surgical Removal.” In some embodiments, the standardized coding scheme 230 includes an ontology that specifies the permitted relationship types between medical concepts, e.g., “is a type of,” “causes,” “is a symptom of,”etc.

In this example, the ontological representation module 124 creates the medical ontological representation 234 based on the standardized coding scheme 230 (e.g., during offline processing). The ontological representation module 124 also maps medical concepts in the standardized coding scheme 230 to concepts in the medical ontological representation 234 (e.g., during run-time operation as the user input/output module 120 receives user requests for instructions). The ontological representation module 124 maps a synonym or metadata for the medical concept in the standardized coding scheme 230 to a property of the concept in the medical ontological representation 234. The ontological representation module 124 maps a relationship between two concepts in the standardized coding scheme 230 to a relationship between the two corresponding concepts in the medical ontological representation 234.

The embedding module 126 generates a medical knowledge vector database 236 from the standardized coding scheme 230. The embedding module 126 converts a medical concept from the standardized coding scheme 230 into an embedding, which is a multi-dimensional vector representing the semantic meaning of one or more words. In general when performing a vector search query, the closer the geometric distance between two embeddings, the greater the semantic similarity. For example, “diabetes” and “insulin” embeddings likely have a higher cosine similarity than “diabetes” and “influenza” embeddings.

The information extraction module 122 processes information from the educational resources repository 150 and/or standard patient education documents 208. The educational resources repository 150 may include electronic medical education materials, such as scanned textbooks, journal articles, conference papers, lectures, clinical practice guidelines, standard operating procedures, known drug interactions, etc. The standard patient education documents 208 may include generic (non-personalized) patient instructions for preparing for or recovering from a medical condition (e.g., for treating the condition, or for recovering from a surgery or other event relating to the medical condition, etc.).

In some embodiments, the information extraction module 122 retrieves the medical education materials from the educational resources repository 150 and/or the standard patient education documents 208. In one embodiment, the information extraction module 122 periodically checks for and retrieves updated medical education materials. In some embodiments, the information extraction module 122 uses OCR to convert scanned documents into text data. In some embodiments, the information extraction module 122 transcribes video and/or audio resources into text data.

In some embodiments, the information extraction module 122 splits the text data into a plurality of text chunks. A text chunk may comprise a word, a sentence, a paragraph, or any other suitable unit of text. In some embodiments, the information extraction module 122 performs named entity recognition (NER) on a given text chunk to recognize a medical concept, such as the name of a condition, disease, prescription drug, etc. The information extraction module 122 applies the standardized coding scheme 230 to the plurality of text chunks to translate the medical concept into a standardized terminology, e.g., “heart attack” may be replaced with “myocardial infarction.”

In this example embodiment, the ontological representation module 124 supplements the medical ontological representation 234 using the standardized text chunks. The ontological representation module 124 may extract a triple of the form “(subject, predicate, object)” from the standardized text chunk, where the predicate describes the relationship between the subject and the object. One example of a triple is (“high sodium intake,” “causes,” “hypertension”). In some embodiments, the ontological representation module 124 sends the text chunk and a triplet extraction query to a language model to generate the triples. Using the generated triples, the ontological representation module 124 adds new concepts and relationships to the medical ontological representation 234 and/or adds new relationships between existing concepts in the medical ontological representation 234. The ontological representation module 124 applies an ontology 232 to specify a concept class for a new concept and a relationship class for a new relationship. The ontology 232 specifies a plurality of concept classes, such as condition, disease, drug, etc., and a plurality of relationship classes, such as is a type of, causes, is a symptom of, etc. The ontological representation module 124 may save the concept class as a property of the concept and may save the relationship class as a property of the relationship in the medical ontological representation 234. The ontological representation module 124 may save source citations as properties for the concept and/or relationship.

The embedding module 126 supplements the medical knowledge vector database 236 with the medical knowledge extracted from the educational resources repository 150 and/or the standard patient education documents 208. The embedding module 126 converts a standardized text chunk into an embedding and adds the embedding to the medical knowledge vector database 236.

FIG. 2C depicts an example process 200C for mapping a patient condition and patient information to a medical ontological representation and a vector database and extracting relevant information. In alternative embodiments, the process 200C includes more, fewer, and/or different components and/or steps than those shown in FIG. 2C. The user input/output module 120 (not depicted) receives an instruction request 238. In some embodiments, the instruction request 238 includes a patient name, patient birthdate or other identifier, and a name of a condition for which the user seeks individual-specific instructions. In some embodiments, the instruction request 238 includes one or more goals, e.g., conditions for which the user seeks interventions. In some embodiments, the user input/output module 120 performs user authentication.

The information extraction module 122 retrieves patient information from the individual records repository 160. In some embodiments, the information extraction module 122 uses OCR to convert scanned documents comprising patient information into text data. In some embodiments, the information extraction module 122 transcribes video and/or audio resources comprising patient information into text data. The information extraction module 122 may periodically check for new or updated patient information in the individual records repository 160. In some embodiments, the information extraction module 122 categorizes the patient information into concepts, allergies, family history, medications, patient, problems, social history, surgical history, and/or vitals.

The information extraction module 122 splits the patient information text data into multiple text chunks and, in some embodiments and/or scenarios, also splits the instruction request 238 into multiple text chunks. In other embodiments and/or scenarios, the information extraction module 122 does not split the instruction request 238 into text chunks. The information extraction module 122 performs NER on one or more of the text chunks to recognize medical terms. The information extraction module 122 applies the standardized coding scheme 230 to the text chunk to translate the medical term into a standardized format.

The ontological representation module 124 generates one or more queries, to query the medical ontological representation 234, using medical terminology obtained from the instruction request 238 and/or the patient information obtained from the individual records repository 160. In some embodiments, the ontological representation module 124 maps a medical term from the instruction request 238 to a corresponding concept in the medical ontological representation 234. In some embodiments, the ontological representation module 124 maps a medical term from the patient information to a corresponding concept of the medical ontological representation 234. In some embodiments, the ontological representation module 124 determines corresponding concepts of the medical ontological representation 234 using text matching of medical terms or numerical matching of standardized medical codes.

In some embodiments, the ontological representation module 124 executes one or more pre-coded queries. In some embodiments, the ontological representation module 124 queries the medical ontological representation 234 to determine the shortest path between two specified concepts (if such a path exists). An exemplary Cypher query may include “MATCH path=SHORTEST 1 (89498:Condition)-[:LINK]-+(87492:Condition) WHERE 89498.name=“diabetes” AND 87492.name=“knee surgery” RETURN [n in nodes(path) AS nodes, length(path) AS pathlength.” In some embodiments, the query excludes a path having a length greater than a specified/predetermined threshold value, e.g., five intervening concepts or six intervening relationships. In some embodiments, the ontological representation module 124 queries a specified concept and returns one or more properties. An exemplary Cypher query may include “MATCH node:Condition {name: ‘diabetes’}] RETURN node.cause.” In some embodiments, the ontological representation module 124 queries a specified concept and returns one or more relationships of the specified concept. An exemplary Cypher query may include “MATCH (node:Condition {name: ‘hypertension’})-[r:TREATMENT]->(m) RETURN m.” In some embodiments, the ontological representation module 124 queries a specified concept and returns and returns one or more properties of the concept.

In some embodiments, the ontological representation module 124 sends a natural language prompt to an LLM instructing the LLM to generate an ontological representation query performing a specified function, e.g., “Generate a Cypher query that returns the shortest path between the concept named ‘diabetes’ and the concept named ‘knee surgery.’” The prompt may be a few-shot prompt that includes exemplary requests and corresponding queries.

The ontological representation module 124 outputs ontological representation information 242, which includes information retrieved from the medical ontological representation 234, such as a path between specified concepts, properties of a specified concept, and/or properties of a relationship. In some embodiments, the ontological representation module 124 provides all or a subset of the retrieved information to the embedding module 126.

The embedding module 126 generates an embedding of a text chunk obtained from the instruction request 238. In some embodiments, the embedding module 126 generates an embedding of the ontological representation information 242 received from the ontological representation module 124. The embedding module 126 queries the medical knowledge vector database 236 for embeddings semantically similar to a specified text embedding. The embedding module 126 retrieves a text chunk corresponding to a semantically similar embedding. In some embodiments, the retrieved text chunk includes a citation to the information source from which the text chunk was obtained. The embedding module 126 outputs the retrieved text chunk as embedding information 244.

FIG. 2D depicts an exemplary process 200D for generating a prompt using RAG, providing the prompt to the generative language model, and receiving individual-specific, relevant output. In alternative embodiments, the process 200D includes more, fewer, and/or different components and/or steps than those shown in FIG. 2D.

The prompt module 128 receives the instruction request 238 and one or more of the following as input: a chat history 252, business rules 256, ontological representation information 242, and embedding information 244. The chat history 252 may include the full text or a summary of one or more chat questions and answers during one or more chat sessions. The business rules 256, such as admission, discharge, and transfer (ADT) rules, include directives to the trained CLaM 132 when generating output. For example, the business rules 256 may specify that instructions are to be no more than one page in length or that if a chat user is experiencing symptoms of a heart attack to respond with a direction to stop the chat and call 911. In some embodiments, the prompt module 128 may summarize and/or reword the instruction request 238, chat history 252, business rules 256, ontological representation information 242, and embedding information 244 when generating the prompt. In some embodiments, the prompt module 128 may use GAR to augment the prompt with the chat history 252, business rules, ontological representation information 242, and/or embedding information 244. In some embodiments, the prompt includes text directed to the trained CLaM 132 (or another suitable generative language model), such as “You are an experienced physician” or “Generate the instructions easily understood by someone with a high school education.” In some embodiments, the prompt includes one or more goals specifying one or more conditions for which the user seeks intervention instructions.

The prompt module 128 submits the prompt to the trained CLaM 132. The trained CLaM 132 generates requested instructions 264. In some embodiments, the requested instructions 264 include citations to one or more sources obtained from the ontological representation information 242. The user input/output module 120 outputs the requested instructions 264 to the user.

### Example Medical Ontological Representation

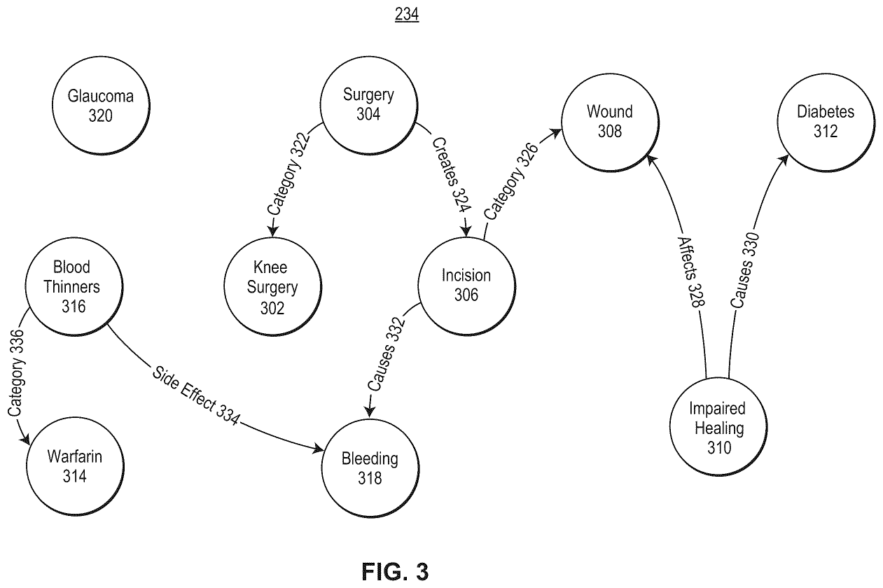

FIG. 3 depicts an example medical ontological representation 234 including concepts 302-320 and relationships 322-334. In alternative embodiments, the medical ontological representation 234 includes more and/or different concepts and/or relationships than those shown in FIG. 3.

In this example, the medical ontological representation 234 depicts ten concepts: knee surgery 302, surgery 304, incision 306, wound 308, impaired healing 310, diabetes 312, warfarin 314, blood thinners 316, bleeding 318, and glaucoma 320. In the medical ontological representation 234, a relationship links two nodes. In the illustrated example, relationship types include category (e.g., category 322 and 336) causes (e.g., causes 324, 330, 332), affects (e.g., affects 328), and side effect (e.g., side effect 334). A relationship may indicate a hierarchical relationship between two concepts, e.g., with knee surgery 302 (node) being a category 322 (edge) of surgery 304 (node).

The ontological representation module 124 may query the medical ontological representation 234 to determine the presence or absence of a direct path (linked by a relationship) or indirect path (linked by intervening concepts and relationships) between two concepts. The presence of a direct or indirect path may indicate a comorbidity. For example, a user may submit a request for instructions for knee surgery (condition) on a patient who has diabetes and glaucoma and takes warfarin (patient information). The ontological representation module 124 determines the corresponding concepts knee surgery 302, diabetes 312, warfarin 314, and glaucoma 320 in the medical ontological representation 234.

The ontological representation module 124 uses inferential reasoning to determine whether the patient information is relevant to, and potentially a comorbidity of, the patient condition. The ontological representation module 124 determines one or more paths from the patient condition concept, knee surgery 302, and the patient information concepts, diabetes 312, warfarin 314, and glaucoma 320. The one or more paths may be a single shortest path or one or more paths satisfying a path criteria, e.g., fewer than four intervening nodes, fewer than five intervening relationships, etc. The ontological representation module 124 uses a depth first search and/or a breadth first search algorithm in some embodiments. For example, the ontological representation module 124 determines that knee surgery 302 is a category of surgery 304, which causes an incision 306, which is a category of wound 308, which is affected by impaired healing 310, which is caused by diabetes 312. Thus, the ontological representation module 124 may determine that diabetes 312 is a relevant comorbidity of knee surgery 302 based on the indirect path between the two concepts.

As another example, the ontological representation module 124, after determining the shortest path between knee surgery 302 and surgery 304 and incision 306, determines that incision 306 causes bleeding 318, which is a side effect of blood thinners 316, of which warfarin 314 is a type. Thus, the ontological representation module 124 determines that use of warfarin 314 is a comorbidity of knee surgery 302 based on the indirect path between the two concepts.

As another example, the ontological representation module 124 attempts to find the shortest path between knee surgery 302 and glaucoma 320. In the medical ontological representation 234, either no path between knee surgery 302 and glaucoma 320 exists, or such a path violates one or more constraints, e.g., maximum number of intervening concepts or relationships, relationship type, etc. Thus, the ontological representation module 124 determines that glaucoma 320 is not a relevant comorbidity of knee surgery 302.

The ontological representation module 124 returns information about the patient condition concept and any patient comorbidity concepts as ontological representation information 242. In some embodiments, the ontological representation information 242 includes properties of the patient condition concept and any patient comorbidity concepts, the intervening concepts and relationships on the determined one or more paths, one or more concepts directly connected to the patient condition concepts and/or the patient comorbidity concepts, or any other relevant information.

### Example Medical Knowledge Vector Database

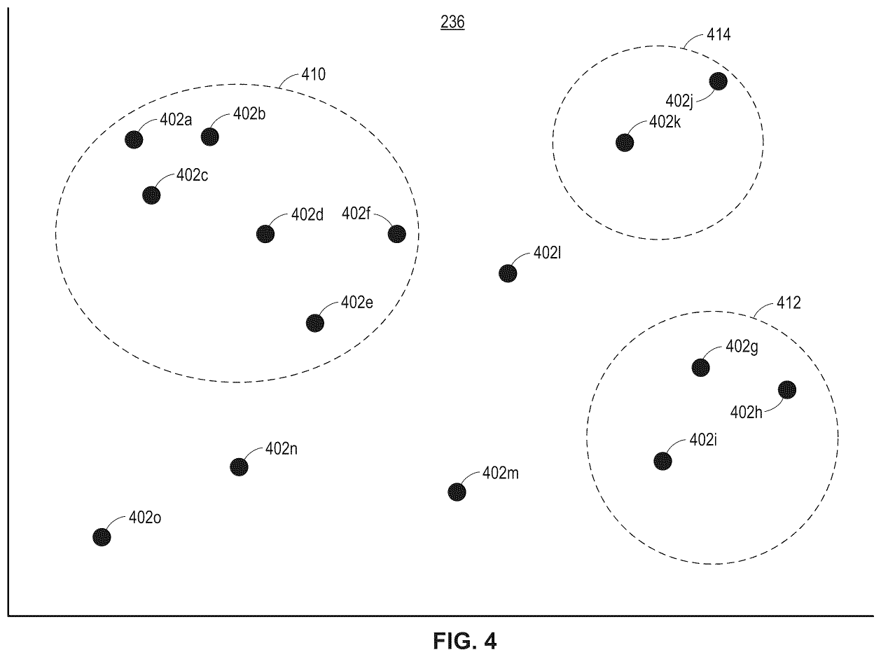

FIG. 4 depicts an example of a medical knowledge vector database 236 including a plurality of vectors/embeddings 402a-402o. In alternative embodiments, the medical knowledge vector database 236 includes more and/or different embeddings than those shown in FIG. 4. An embedding corresponds to a text chunk comprising a medical concept. Although FIG. 4 depicts the medical knowledge vector database 236 in two-dimensional space, the embedding may have any suitable number (e.g., hundreds or thousands) of dimensions.

The embedding module 126 maps the patient condition to an embedding and maps the patient comorbidity to another embedding. For example, embedding 402a may represent “knee surgery,” embedding 402g may represent “diabetes,” and embedding 402j may represent “warfarin.” Thus, the embedding module may map the patient condition to embedding 402a and map the patient comorbidities to embeddings 402g and 402j.

The embedding module 126 searches for any additional embeddings semantically similar to the condition embeddings and/or any additional embeddings semantically similar to the comorbidity embeddings. The embedding module 126 performs the vector search query by calculating the geometric distance between two embeddings, e.g., via cosine similarity, Euclidean distance, etc., and determining the closest embeddings. In some embodiments, the embedding module 126 performs a clustering algorithm, e.g., k-means, to partition the embeddings into clusters of semantically similar embeddings. As an example, embedding 402b may represent “After knee surgery, apply ice to the knee for 24 hours,” embedding 402c may represent “After knee surgery, keep the knee elevated,” embedding 402d may represent “Do not drive or operate heavy machinery for 24 hours after receiving anesthesia,” embedding 402e may represent “Keep your incision clean and bandaged,” and embedding 402f may represent “Take your pain medication as directed by your physician.” The embedding module 126 may cluster embeddings 402a-402f into a surgery cluster 410. Embedding 402h may represent “Diabetic patients should monitor their blood sugar levels regularly,” and embedding 402i may represent “Diabetic patients should limit their dietary sugar intake.” The embedding module 126 may cluster embeddings 402g-402i into a diabetes cluster 412. Embedding 402k may represent “Stop taking blood thinners five days before surgery.” The embedding module 126 may cluster embeddings 402j and 402k into a blood thinner cluster 414. The embedding module 126 may determine that embeddings 402l-402o are insufficiently similar to embeddings 402a, 402g, and 402j and thus does not include them in the clusters 410, 412, and 414.

The embedding module 126 outputs the text associated with the semantically similar additional embeddings as the embedding information 244.

### Example Method of Generating Instructions

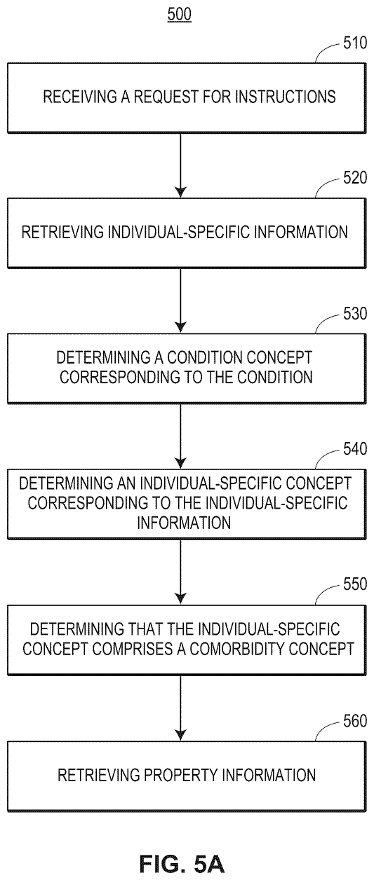

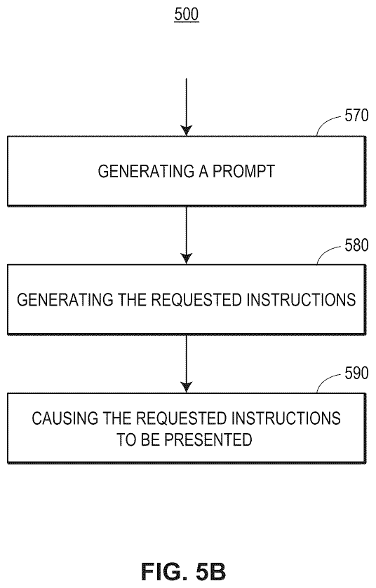

FIGS. 5A and 5B depict a flow diagram of an exemplary computer-implemented method 500 for generating individual-specific instructions. One or more (e.g., all) steps of the computer-implemented method 500 are implemented as a set of instructions stored on a computer-readable memory and executable on one or more processors. In some embodiments, the computer-implemented method 500 of FIGS. 5A and 5B is implemented via a system, such as the server 110, educational resources repository 150, individual records repository 160, and/or the client device 170. The computer-implemented method 500 may operate in conjunction with one or more of the scenarios and/or environments illustrated in FIGS. 1-4 and/or in other environments. In some embodiments, for example, the computer-implemented method 500 is repeated as necessary, such as when a patient's condition is updated or a patient submits a new question.

As illustrated in FIG. 5A, the example computer-implemented method 500 includes at block 510 receiving a request for instructions. In some embodiments, the user input/output module 120 receives the request for instructions from the client device 170 via a chat session, web form, application 180, e-mail message, etc. In some embodiments, the request for instructions comprises a chat question. In some embodiments, the request for instructions relates to a condition of an individual.

The example computer-implemented method 500 includes at block 520 retrieving individual-specific information from one or more records associated with the individual. The information extraction module 122 retrieves the individual-specific information from the system database 134, individual records repository 160, or another source. The information extraction module 122 retrieves individual-specific information, such as medical records and/or medical insurance claims, including health summaries, healthcare utilization data, claims records, prescriptions, diagnoses, laboratory test results, risk adjustment factor (RAF) score or other risk scores, vital sign information, past hospitalizations and surgeries, etc. In some embodiments, the information extraction module 122 converts scanned documents into text data using OCR and/or transcribes audio and/or video into text data. The information extraction module 122 splits text data into a plurality of text chunks. The information extraction module 122 performs NER to determine/extract the names of diseases, conditions, procedures, symptoms, prescriptions, etc. In some embodiments, the information extraction module 122 applies a coding scheme, such as FHIR, SNOMED-CT, LOINC, or ICD, to translate the extracted names of diseases, conditions, procedures, symptoms, prescriptions, etc. into a standardized format, such as a formal medical name or an alphanumeric code.

The example computer-implemented method 500 includes at block 530 determining a condition concept corresponding to the condition and is represented in the ontological representation. In some embodiments, the ontological representation module 124 performs a keyword search of the condition name or alphanumeric code in the ontological representation to find a corresponding condition concept. In some embodiments, the condition concept corresponding to the condition information is determined using NER. In some embodiments, the ontological representation represents a plurality of concepts and a plurality of relationships linking different concepts of the plurality of concepts. In some embodiments, the condition concept and the individual-specific concept are normalized by applying a standardized coding scheme

The example computer-implemented method 500 includes at block 540 determining the individual-specific concept corresponding to the individual-specific information and is represented in the ontological representation. In some embodiments, the ontological representation module 124 performs a keyword search of the name or alphanumeric code associated with the individual-specific information in the ontological representation to find a corresponding individual-specific concept. In some embodiments, the individual-specific concept corresponding to the individual-specific information is determined using NER.

The example computer-implemented method 500 includes at block 550 determining that the individual-specific concept comprises a comorbidity concept relevant to the condition concept. In some embodiments, determining that the individual-specific concept comprises a comorbidity concept includes the ontological representation module 124 searching for a path linking the condition concept to the individual-specific concept in the ontological representation, where the existence of the path indicates that the individual-specific concept comprises a comorbidity concept. A path includes the condition concept, the individual-specific concept, and one or more intervening concepts of the plurality of concepts and one or more intervening relationships of the plurality of relationships that collectively link the condition concept to the individual-specific concept. The ontological representation module 124 searches for the path using a graph traversal algorithm, such as breadth first search or depth first search. In some embodiments, the path must satisfy specified criteria, such as relationship types, maximum intervening concepts, maximum intervening relationships, etc.

As illustrated in FIG. 5B, the example computer-implemented method 500 includes at block 560 retrieving property information including condition property data associated with the condition concept, and individual-specific property data associated with the comorbidity concept. In some embodiments, the ontological representation module 124 retrieves intervening property data associated with the one or more intervening concepts in the path.

The example computer-implemented method 500 includes at block 570 generating a prompt based on the request for the instructions and the property information. The prompt module 128 uses RAG to include the property information in the prompt. In some embodiments, the prompt module 128 may generate the prompt based on a chat history associated with the user, business rules, and/or the embedding information. In some embodiments, the prompt module 128 includes directives to the generative language model in the prompt. The directives may include the educational level of the individual for whom the instructions are requested.

The example computer-implemented method 500 includes at block 580 generating the requested instructions. The prompt module 128 may provide the prompt as input to a generative language model, such as the trained CLaM 132. The generative language model outputs the requested instructions in response to receiving the prompt. In some embodiments, the requested instructions are generated in the form of a chat answer. In some embodiments, the requested instructions include the citation to the one or more educational resources. In some embodiments, the generative language model generates text corresponding to the educational level specified in the prompt.

The example computer-implemented method 500 includes at block 590 causing the requested instructions to be presented on a display. The user input/output module 120 presents the requested instructions on a display associated with the client device 170 or the server 110.

In some embodiments, the example computer-implemented method 500 includes training the generative language model using one or more training documents comprising domain-specific information. In some embodiments, the training module 130 tunes the generative language model using reinforcement learning. Reinforcement learning may include human and/or machine generated scoring of generated language.

In some embodiments, the example computer-implemented method 500 includes retrieving a first concept, a second concept, and a relationship from a standardized coding scheme, where the relationship specifies a link between the first concept and the second concept. In some embodiments, the information extraction module 122 uses NER to extract the first concept, second concept, and the relationship from the standardized coding scheme. In some embodiments, the information extraction module 122 submits the standardized coding scheme to a language model and receives the first concept, second concept, and relationship as output. In some embodiments, the information extraction module 122 retrieves property information associated with the first concept, property information associated with the second concept, and property information associated with the relationship. The property information may include a citation to one or more educational resources associated with the first concept, second concept, and/or relationship. The standardized coding scheme may include FHIR, SNOMED-CT, LOINC, ICD, etc. The ontological representation module 124 adds the first concept, second concept, and relationship to the ontological representation. In some embodiments, the embedding module 126 generates a first knowledge base embedding based on the first concept and a second knowledge base embedding based on the second concept. The embedding module 126 saves the first knowledge base embedding and the second knowledge base embedding in a vector database.

In some embodiments, the example computer-implemented method 500 includes retrieving text from one or more educational resources. The information extraction module 122 retrieves the individual-specific information from the system database 134, educational resources repository 150, or another source. In some embodiments, the information extraction module 122 converts scanned documents into text data using OCR and/or transcribes audio and/or video into text data. The information extraction module 122 splits text data into a plurality of text chunks. The information extraction module 122 performs NER to determine a first concept, a second concept, and a relationship in the text. In some embodiments, the information extraction module 122 applies a standardized coding scheme, such as FHIR, SNOMED-CT, LOINC, or ICD, to normalize the first concept, the second concept, and the relationship. The ontological representation module 124 adds the normalized first concept, second concept, and relationship to the ontological representation. In some embodiments, the embedding module 126 generates a first knowledge base embedding based on the first concept and a second knowledge base embedding based on the second concept. The embedding module saves the first knowledge base embedding and the second knowledge base embedding in the vector database.

In some embodiments, the example computer-implemented method 500 includes determining a condition embedding that corresponds to the condition property data. The embedding module 126 determines the condition embedding in a vector database comprising a plurality of knowledge base embeddings. The embedding module 126 determines a comorbidity embedding in the vector database, where the comorbidity embedding corresponds to the individual-specific property data associated with the comorbidity concept. The embedding module 126 retrieves embedding information similar to the corresponding condition embedding and/or the corresponding comorbidity embedding. In some embodiments the embedding module 126 uses geometric distance, such as Euclidean distance or cosine similarity, to determine which embeddings are most semantically similar to the condition embedding and/or comorbidity embedding. The embedding module 126 retrieves the embedded information corresponding to the semantically similar embeddings.

It should be understood that not all blocks of the computer-implemented method 500 are required to be performed. Moreover, the computer-implemented method 500 is not mutually exclusive (i.e., block(s) from computer-implemented method 500 may be performed in any particular implementation). Further, in some embodiments, the operations of the computer-implemented method 500 are not performed strictly in the order shown in FIGS. 5A and 5B.

### EXAMPLES

### Example 1

A computer-implemented method comprising: receiving, by one or more processors, a request for instructions relating to a condition of an individual; retrieving, by the one or more processors, individual-specific information from one or more records associated with the individual; determining, by the one or more processors, a condition concept that corresponds to the condition and is represented in an ontological representation, the ontological representation representing (i) a plurality of concepts, and (ii) a plurality of relationships linking different concepts of the plurality of concepts; determining, by the one or more processors, an individual-specific concept that corresponds to the individual-specific information and is represented in the ontological representation; determining, by the one or more processors and using the ontological representation, that the individual-specific concept comprises a comorbidity concept relevant to the condition concept; retrieving, by the one or more processors, property information comprising (i) condition property data associated with the condition concept, and (ii) individual-specific property data associated with the comorbidity concept; generating, by the one or more processors, a prompt based on (i) the request for the instructions, and (ii) the property information; generating, by the one or more processors, requested instructions, at least in part by providing the prompt as an input to a generative language model; and causing, by the one or more processors, the requested instructions to be presented on a display.

### Example 2

The computer-implemented method of Example 1, wherein determining that the individual-specific concept comprises the comorbidity concept comprises: searching for a path linking the condition concept to the individual-specific concept in the ontological representation, wherein the path comprises (i) the condition concept, (ii) the individual-specific concept, and (iii) one or more intervening concepts of the plurality of concepts and one or more intervening relationships of the plurality of relationships that collectively link the condition concept to the individual-specific concept.

### Example 3

The computer-implemented method of Example 2, wherein retrieving the property information further comprises: retrieving intervening property data associated with the one or more intervening concepts.

### Example 4

The computer-implemented method of any of Examples 1 through 3, further comprising: determining, by the one or more processors, a condition embedding in a vector database comprising a plurality of knowledge base embeddings, wherein the condition embedding corresponds to the condition property data; determining, by the one or more processors, a comorbidity embedding in the vector database, wherein the comorbidity embedding corresponds to the individual-specific property data associated with the comorbidity concept; and retrieving, by the one or more processors, embedding information semantically similar to (i) the corresponding condition embedding, and/or (ii) the corresponding comorbidity embedding; wherein generating the prompt is further based on the embedding information.

### Example 5

The computer-implemented method of Example 4, further comprising: retrieving, by the one or more processors, a first concept, a second concept, and a relationship from a standardized coding scheme, wherein the relationship specifies a path link between the first concept and the second concept; adding, by the one or more processors, the first concept, the second concept, and the relationship to the ontological representation; generating, by the one or more processors and based on the first concept, a first knowledge base embedding of the plurality of knowledge base embeddings; and generating, by the one or more processors and based on the second concept, a second knowledge base embedding of the plurality of knowledge base embeddings.

### Example 6

The computer-implemented method of Example 4 or Example 5, further comprising: retrieving, by the one or more processors, text from one or more educational resources; determining, by the one or more processors, a first concept, a second concept, and a relationship in the text using named entity recognition, wherein the relationship specifies a path link between the first concept and the second concept; normalizing, by the one or more processors, the first concept, the second concept, and the relationship by applying a standardized coding scheme; adding, by the one or more processors, the first concept, the second concept, and the relationship to the ontological representation; generating, by the one or more processors and based on the first concept, a first knowledge base embedding of the plurality of knowledge base embeddings; and generating, by the one or more processors and based on the second concept, a second knowledge base embedding of the plurality of knowledge base embeddings.

### Example 7

The computer-implemented method of Example 6, further comprising: adding, by the one or more processors, citation property information to the first concept, the second concept, and/or the relationship, wherein the citation property information comprises a citation to a respective one of the one or more educational resources, wherein generating the requested instructions comprises generating including a the citation to a respective one of the one or more educational resource sin the requested instructions

### Example 8

The computer-implemented method of any of Examples 1 through 7, wherein determining the condition concept that corresponds to the condition information and determining the individual-specific concept that corresponds to the individual-specific information comprise: determining the condition concept that corresponds to the condition information, and determining the individual-specific concept that corresponds to the individual-specific information, using named entity recognition; and normalizing, by the one or more processors, the condition concept and the individual-specific concept by applying a standardized coding scheme.

### Example 9

The computer-implemented method of any of Examples 1 through 8, wherein generating the prompt is further based on a specified education level of the individual, and wherein generating the requested instructions comprises generating text corresponding to the specified education level.

### Example 10

The computer-implemented method of any of Examples 1 through 9, further comprising: training, by the one or more processors, the generative language model using one or more training documents comprising domain-specific information; and tuning, by the one or more processors, the generative language model using reinforcement learning.

### Example 11

The computer-implemented method of any of Examples 1 through 10, wherein the request for instructions comprises a chat question, wherein generating the prompt comprises generating the prompt based on a chat history, and wherein generating the requested instructions comprises generating a chat answer.

### Example 12

A system comprising memory and one or more processors communicatively coupled to the memory, the one or more processors configured to perform the method of any one of Examples 1 through 11.

### Example 13

One or more non-transitory computer-readable storage media including instructions that, when executed by one or more processors, cause the one or more processors to perform the method of any of Examples 1 through 11.

### Additional Considerations

Throughout this specification, components, operations, or structures described as a single instance may be implemented as multiple instances. Although individual operations of one or more methods (or processes, techniques, routines, etc.) are illustrated and described as separate operations, two or more of the individual operations may be performed concurrently or otherwise in parallel, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of routines, subroutines, applications, or instructions. These may constitute and/or be implemented by software (e.g., code embodied on a non-transitory, machine-readable medium), hardware, or a combination thereof. In hardware, the routines, etc., may represent tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware component that operates to perform certain operations as described herein.

In various embodiments, a hardware component may be implemented mechanically or electronically. For example, a hardware component may comprise dedicated circuitry or logic that is permanently configured (e.g., as a special-purpose processor, such as a field programmable gate array (FPGA) or an application-specific integrated circuit (ASIC)) to perform certain operations. A hardware component may also or instead comprise programmable logic or circuitry (e.g., as encompassed within one or more general-purpose processors and/or other programmable processor(s)) that is temporarily configured by software to perform certain operations.

Accordingly, the term “hardware component” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where the hardware components include a general-purpose processor configured using software, the general-purpose processor may be configured as respective different hardware components at different times. Software may accordingly configure a processor, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple of such hardware components exist contemporaneously, communications may be achieved through signal transmission (e.g., over appropriate circuits and buses) that connect the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information).

As noted above, the various operations of example methods (or processes, techniques, routines, etc.) described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions. The components referred to herein may, in some example embodiments, comprise processor-implemented components.

Moreover, the performance of certain of the operations may be distributed among the one or more processors, not only residing within a single machine, but deployed across a number of machines. In some example embodiments, the one or more processors or processor-implemented components may be located in a single geographic location (e.g., within a home environment, an office environment, or a server farm). In other example embodiments, the one or more processors or processor-implemented modules may be distributed across a number of geographic locations.

Unless otherwise apparent from the context of use, reference in the present disclosure to a same set of “one or more processors” (or a same “plurality of processors,” etc.) performing multiple operations can encompass implementations in which performance of the operations is divided among the processor(s) in any suitable way. For example, “generating, by one or more processors, X; and generating, by the one or more processors, Y” can encompass: (1) implementations in which a first subset of the processors (e.g., in a first computing device) generates X and an entirely distinct, second subset of the processors (e.g., in a different, second computing device) independently generates Y; (2) implementations in which all of the processor(s) (e.g., one or multiple processors in the same device, or multiple processors distributed among multiple devices) contribute to the generation of both X and Y; and (3) other variations.

Moreover, each operation of processes illustrated as logical flow graphs may represent a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be combined in any order and/or in parallel to implement the processes.

Any discussion of receiving data associated with an individual that may be protected, confidential, or otherwise sensitive information, can be understood to have been preceded by transmitting a notice of use of the data, receiving authorization to use the data, and/or providing a mechanism by which a user may cause use of the data to cease or a copy of the data to be provided to the user.

The terms “coupled” and “connected,” along with their derivatives, may be used. In particular embodiments, “connected” may be used to indicate that two or more elements are in direct physical or electrical contact with each other, although the context in the description may dictate otherwise when it is apparent that two or more elements are not in direct physical or electrical contact. “Coupled” may mean that two or more elements are in direct physical or electrical contact. However, “coupled” may also mean that two or more elements are not in direct contact with each other, yet still co-operate, transmit between, or interact with each other.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “some embodiments,” “one embodiment,” “an embodiment,” or the like means that a particular element, feature, structure, or characteristic described in connection with the embodiment is included in at least one embodiment, but not every embodiment necessarily includes the particular element, feature, structure, or characteristic. Different instances of the phrase “in one embodiment” in various places in the specification do not necessarily all refer to the same embodiment, although they may in some cases.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless the context of use clearly indicates otherwise, “or” refers to an inclusive or and not to an exclusive or. For example, a condition A or B is satisfied by any one of the following: A is true (or present) and B is false (or not present), A is false (or not present) and B is true (or present), and both A and B are true (or present).

For the purposes of the present disclosure, the term “a” or “an” entity refers to one or more of that entity. As such, the terms “a” or “an”, “one or more”, and “at least one” can be used interchangeably herein unless explicitly contradicted by the specification using the word “only one” or similar. For example, “a first element” may functionally be interpreted as “a first one or more elements” or a “first at least one element.” The term “set” is intended to mean a collection of elements and can be a null set (i.e., a set containing zero elements) or may comprise one, two, or more elements. A “subset” is intended to mean a collection of elements that are all elements of a set, but that does not include other elements of the set. A first subset of a set may comprise zero, one, or more elements that are also elements of a second subset of the set. The first subset may be said to be a subset of the second subset if all the elements of the first subset are elements of the second subset, while also being a subset of the set. However, if all the elements of the second subset are also elements of the first subset (in addition to all the elements of the first subset being elements of the second subset), the first subset and the second subset are a single subset/not distinct.

Upon reading this disclosure, those of skill in the art will appreciate still additional alternative structural and functional designs through the principles disclosed herein. Therefore, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A computer-implemented method comprising:
receiving, by one or more processors, a request for instructions relating to a condition of an individual;
retrieving, by the one or more processors, individual-specific information from one or more records associated with the individual;
determining, by the one or more processors, a condition concept that corresponds to the condition and is represented in an ontological representation, the ontological representation representing (i) a plurality of concepts, and (ii) a plurality of relationships linking different concepts of the plurality of concepts;
determining, by the one or more processors, an individual-specific concept that corresponds to the individual-specific information and is represented in the ontological representation;
determining, by the one or more processors and using the ontological representation, that the individual-specific concept comprises a comorbidity concept relevant to the condition concept;
retrieving, by the one or more processors, property information comprising (i) condition property data associated with the condition concept, and (ii) individual-specific property data associated with the comorbidity concept;
generating, by the one or more processors, a prompt based on (i) the request for the instructions, and (ii) the property information;
generating, by the one or more processors, requested instructions, at least in part by providing the prompt as an input to a generative language model; and
causing, by the one or more processors, the requested instructions to be presented on a display.

2. The computer-implemented method of claim 1, wherein determining that the individual-specific concept comprises the comorbidity concept comprises:
searching for a path linking the condition concept to the individual-specific concept in the ontological representation, wherein the path comprises (i) the condition concept, (ii) the individual-specific concept, and (iii) one or more intervening concepts of the plurality of concepts and one or more intervening relationships of the plurality of relationships that collectively link the condition concept to the individual-specific concept.

3. The computer-implemented method of claim 2, wherein retrieving the property information further comprises:
retrieving intervening property data associated with the one or more intervening concepts.

4. The computer-implemented method of claim 1, further comprising:
determining, by the one or more processors, a condition embedding in a vector database comprising a plurality of knowledge base embeddings, wherein the condition embedding corresponds to the condition property data;
determining, by the one or more processors, a comorbidity embedding in the vector database, wherein the comorbidity embedding corresponds to the individual-specific property data associated with the comorbidity concept; and
retrieving, by the one or more processors, embedding information semantically similar to (i) the corresponding condition embedding, and/or (ii) the corresponding comorbidity embedding;
wherein generating the prompt is further based on the embedding information.

5. The computer-implemented method of claim 4, further comprising:
retrieving, by the one or more processors, a first concept, a second concept, and a relationship from a standardized coding scheme, wherein the relationship specifies a link between the first concept and the second concept;
adding, by the one or more processors, the first concept, the second concept, and the relationship to the ontological representation;
generating, by the one or more processors and based on the first concept, a first knowledge base embedding of the plurality of knowledge base embeddings; and
generating, by the one or more processors and based on the second concept, a second knowledge base embedding of the plurality of knowledge base embeddings.

6. The computer-implemented method of claim 4, further comprising:
retrieving, by the one or more processors, text from one or more educational resources;
determining, by the one or more processors, a first concept, a second concept, and a relationship in the text using named entity recognition, wherein the relationship specifies a link between the first concept and the second concept;
normalizing, by the one or more processors, the first concept, the second concept, and the relationship by applying a standardized coding scheme;
adding, by the one or more processors, the first concept, the second concept, and the relationship to the ontological representation;
generating, by the one or more processors and based on the first concept, a first knowledge base embedding of the plurality of knowledge base embeddings; and
generating, by the one or more processors and based on the second concept, a second knowledge base embedding of the plurality of knowledge base embeddings.

7. The computer-implemented method of claim 6, further comprising:
adding, by the one or more processors, citation property information to the first concept, the second concept, and/or the relationship, wherein the citation property information comprises a citation to a respective one of the one or more educational resources,
wherein generating the requested instructions comprises including the citation in the requested instructions.

8. The computer-implemented method of claim 1, wherein determining the condition concept that corresponds to the condition information and determining the individual-specific concept that corresponds to the individual-specific information comprise:
determining the condition concept that corresponds to the condition information, and determining the individual-specific concept that corresponds to the individual-specific information, using named entity recognition; and
normalizing, by the one or more processors, the condition concept and the individual-specific concept by applying a standardized coding scheme.

9. The computer-implemented method of claim 1, wherein generating the prompt is further based on a specified education level of the individual, and wherein generating the requested instructions comprises generating text corresponding to the specified education level.

10. The computer-implemented method of claim 1, further comprising:
training, by the one or more processors, the generative language model using one or more training documents comprising domain-specific information; and
tuning, by the one or more processors, the generative language model using reinforcement learning.

11. The computer-implemented method of claim 1, wherein the request for instructions comprises a chat question, wherein generating the prompt comprises generating the prompt based on a chat history, and wherein generating the requested instructions comprises generating a chat answer.

12. A system comprising:
one or more processors; and
a memory storing computer-readable instructions that, when executed by the one or more processors, cause the one or more processors to:
receive a request for instructions relating to a condition of an individual,
retrieve individual-specific information from one or more records associated with the individual,
determine a condition concept that corresponds to the condition and is represented in an ontological representation, the ontological representation representing (i) a plurality of concepts, and (ii) a plurality of relationships linking different concepts of the plurality of concepts,
determine an individual-specific concept that corresponds to the individual-specific information and is represented in the ontological representation,
determine, using the ontological representation, that the individual-specific concept comprises a comorbidity concept relevant to the condition concept,
retrieve property information comprising (i) condition property data associated with the condition concept, and (ii) individual-specific property data associated with the comorbidity concept,
generate a prompt based on (i) the request for the instructions, and (ii) the property information,
generate requested instructions, at least in part by providing the prompt as an input to a generative language model, and
cause the requested instructions to be presented on a display.

13. The system of claim 12, wherein determining that the individual-specific concept comprises the comorbidity concept comprises:
searching for a path linking the condition concept to the individual-specific concept in the ontological representation, wherein the path comprises (i) the condition concept, (ii) the individual-specific concept, and (iii) one or more intervening concepts of the plurality of concepts and one or more intervening relationships of the plurality of relationships that collectively link the condition concept to the individual-specific concept.

14. The system of claim 13, wherein retrieving the property information further comprises:
retrieving intervening property data associated with the one or more intervening concepts.

15. The system of claim 12, wherein the computer-readable instructions further cause the one or more processors to:
determine a condition embedding in a vector database comprising a plurality of knowledge base embeddings, wherein the condition embedding corresponds to the condition property data,
determine a comorbidity embedding in the vector database, wherein the comorbidity embedding corresponds to the individual-specific property data associated with the comorbidity concept, and
retrieve embedding information semantically similar to (i) the corresponding condition embedding, and/or (ii) the corresponding comorbidity embedding;
wherein generating the prompt is further based on the embedding information.

16. The system of claim 15, wherein the computer-readable instructions further cause the one or more processors to:
retrieve a first concept, a second concept, and a relationship from a standardized coding scheme, wherein the relationship specifies a link between the first concept and the second concept,
add the first concept, the second concept, and the relationship to the ontological representation,
generate, based on the first concept, a first knowledge base embedding of the plurality of knowledge base embeddings, and
generate, based on the second concept, a second knowledge base embedding of the plurality of knowledge base embeddings.

17. The system of claim 15, wherein the computer-readable instructions further cause the one or more processors to:
retrieve text from one or more educational resources,
determine a first concept, a second concept, and a relationship in the text using named entity recognition, wherein the relationship specifies a link between the first concept and the second concept,
normalize the first concept, the second concept, and the relationship by applying a standardized coding scheme,
add the first concept, the second concept, and the relationship to the ontological representation,
generate, based on the first concept, a first knowledge base embedding of the plurality of knowledge base embeddings, and
generate, based on the second concept, a second knowledge base embedding of the plurality of knowledge base embeddings.

18. The system of claim 17, wherein the computer-readable instructions further cause the one or more processors to:
add citation property information to the first concept, the second concept, and/or the relationship, wherein the citation property information comprises a citation to a respective one of the one or more educational resources,
wherein generating the requested instructions comprises including the citation in the requested instructions.

19. The system of claim 12, wherein determining the condition concept that corresponds to the condition information and determining the individual-specific concept that corresponds to the individual-specific information comprise:
determining the condition concept that corresponds the condition information, and determining the individual-specific concept that corresponds to the individual-specific information, using named entity recognition, and
normalizing the condition concept and the individual-specific concept by applying a standardized coding scheme.

20. A computer readable storage medium storing non-transitory computer-readable instructions that, when executed on one or more processors, cause the one or more processors to:
receive a request for instructions relating to a condition of an individual;
retrieve individual-specific information from one or more records associated with the individual;
determine a condition concept that corresponds to the condition and is represented in an ontological representation, the ontological representation representing (i) a plurality of concepts, and (ii) a plurality of relationships linking different concepts of the plurality of concepts;
determine an individual-specific concept that corresponds to the individual-specific information and is represented in the ontological representation;
determine, using the ontological representation, that the individual-specific concept comprises a comorbidity concept relevant to the condition concept;
retrieve property information comprising (i) condition property data associated with the condition concept, and (ii) individual-specific property data associated with the comorbidity concept;
generate a prompt based on (i) the request for the instructions, and (ii) the property information;
generate requested instructions, at least in part by providing the prompt as an input to a generative language model; and
cause the requested instructions to be presented on a display.
